(19) United States
(12) Patent Application Publication
(10) Pub. No.: US 2024/0013892 A1
(43) Pub. Date: Jan. 11, 2024

(54) SYSTEM AND METHOD FOR MONITORING THE HABITS OF A USER

(71) Applicant: Emanuele SAURWEIN, Lugano (CH)

(72) Inventor: Emanuele SAURWEIN, Lugano (CH)

(21) Appl. No.: 18/041,555

(22) PCT Filed: Aug. 4, 2021
(86) PCT No.: PCT/IB2021/057127

§ 371 Date: Feb. 14, 2023

(30) Foreign Application Priority Data
Aug. 17, 2020 (IT) 102020000020197

(51) Int. Cl. G16H 20/60; A61B 5/00

(52) U.S. Cl. CPC G16H 20/60; A61B 5/6802; A61B 5/7264

(57) ABSTRACT

A system for monitoring the habits of one or more users which permits sending optimized instructions from both a dietary and a habitation point of view in order to measure and count in a more precise and efficient way the real energy need of an individual within the target of 2000 W society. The provision of the optimized instructions includes a combined analysis of the dwelling's energy production data, the users' nutritional information as well as the constant analysis of their physiological parameters corresponding to both movement data and the liquid and solid evacuations produced by the user over time.

### TECHNICAL FIELD

The present invention relates to a system and method for monitoring the habits of a user. In more detail, the system and method of the invention are also intended to generate a set of food and housing instructions in an optimized form for the user.

### BACKGROUND ART

From the post-war period onwards, energy requirements have grown in proportion to the increase in population density. FIG. 1 shows a graph representing the development of the average energy requirement E (calculated in Watts per person) over the generations since the 1940s. As can be seen, the per capita energy requirement E at the time of this application has tripled to averages of over 6000 W in industrialized countries. An average energy requirement E of 2000 W per capita is equal to the capacity of planet Earth to sustain and regenerate itself Any number below 1 therefore identifies sustainable human consumption, any number above 1 implies unsustainability which increases as the value increases (e.g. a value of 3, equal to 6000 W, implies that three planets would be needed if the entire world population continued to consume as it does today).

In the light of the drastic climate change taking place, a number of international agreements aim to reduce per capita energy use to sustainable averages without changing people's quality of life. One such agreement is that developed by the Swiss Federal Institute of Technology in Zurich and called the “2000 Watt Society”, which aims to reduce the energy needs of each individual to a maximum of 2000 W and 1 tonne of energy per person of CO₂ by 2100. The aim is to count not only the personal energy used—or the dwelling energy consumed—but also the grey energy, i.e. the amount needed to produce, transport and dispose of a product/service or material.

In this context, the design of future dwellings also plays a key role in the 2000 W society project. Many intelligent housing solutions are known in which energy is produced using exclusively renewable sources combined with the use of improved home automation in order to increase the overall energy efficiency of the dwelling unit. However, although these solutions improve in many respects and aim to increase self-sufficiency, they are in fact mostly confined to automated energy management of the dwelling without actually caring about the health and long-term energy balance of the occupants.

In the light of the objectives set out above and the attempts at energy improvement developed by the prior art, the Applicant has realized that in this historical period of constant climatic deterioration, it is no longer sufficient to optimize only the envelope and/or the energy efficiency of the dwelling. In a broader vision of an even more sustainable energy balance, it is necessary to focus more on the inhabitant and his or her behavior. The habits of human beings, their way of living, their health and mobility are therefore extremely decisive in calculating the overall energy balance, their well-being in general, in order to achieve a real climate improvement.

The Applicant has therefore decided to develop a system that will make it possible to monitor not only the energy values of the buildings but also the behavior of the users who live in them as part of an extremely high-performance energy balance project that will, at the same time, have a profound effect on the way future homes are built. In particular, the monitoring of habits will make it possible, in a predefined time frame, to instruct the user on new optimized ways of living, moving and eating, acting within the framework of food self-sufficiency related to the building.

### DESCRIPTION OF THE INVENTION

By virtue of the above, one of the objects of the present invention relates to a system for monitoring the habits of a user which permits sending optimized instructions from both a dietary and a habitation point of view in order to measure and count in a more precise and efficient way the real energy need of an individual within the target of 2000 W society.

The Applicant also thought that the provision of the optimized instructions should include a combined analysis of the dwelling's energy production data, the users' nutritional information as well as the constant analysis of their physiological parameters corresponding to both movement data and the liquid and solid evacuations produced by the user over time.

By virtue of the foregoing considerations, this and other objects of the present invention are achieved by the system for monitoring the habits of a user according to claim 1.

Another object of the present invention is to devise a method for monitoring the habits of a user and to generate a plurality of optimized dietary and/or habitation instructions comprising the use of one or more neural networks according to claim 7.

### BRIEF DESCRIPTION OF THE DRAWINGS

Further characteristics and advantages of the system and method according to the present invention will result from the description below of preferred embodiments thereof, given by way of an indicative yet non-limiting example, with reference to the attached figures, wherein:

FIG. 1 shows a graph representing the development of the per capita energy demand in the period between 1940 and 2019, and the energy target since 2050 onwards (Sources: SUPSI, Swiss Federal Office for Spatial Development “ARE”, 2000 W Society, USTAT);

FIGS. 2-4 show exterior perspective views of the dwelling unit associated with the system of the present invention;

FIGS. 5-8 show interior perspective views of the dwelling unit associated with the system of the present invention;

FIG. 9 shows a representative diagram of the parameters acquired and processed by the processing means and by the neural network of the present invention.

### EMBODIMENTS OF THE INVENTION

With reference to the attached illustrations, U is globally referred to as a user meant to inhabit a dwelling D and to be monitored by the system of the present invention. As will be seen in the remainder of the present disclosure, some embodiments of the present invention have the objective of instructing and helping the user U (or possibly several users U of the same dwelling D) in different areas, such as, e.g.:
the automated and independent management of the dwelling D from a heating, electrical power supply and water point of view,
the feeding and monitoring of the consumption of the user points installed in the dwelling,
the monitoring of one or more vegetable crops intended for food production,
the monitoring of the user's physiological parameters,
the monitoring of the user's nutritional parameters,
the monitoring of the user's mobility-related consumption,
the monitoring of the grey energy for the construction of the dwelling.

As will be seen below, the instructions and suggestions as well as the household automations that will be provided by the system of the invention will be used for the purpose of calculating the overall energy balance of the user during his or her daily life, outside and inside his or her dwelling, in order to maintain as much as possible a lifestyle that allows an average annual energy demand of about 2000 W within the target of 2000 W Society.

Globally, the energy requirements and CO₂ emissions of the dwelling D as well as of the user U are set to meet the limit target values, preferably by the date 2100, of approximately 2000 W per capita. The dwelling D should have the highest possible degree of self-sufficiency compatible with the need to limit the energy used during its construction (so-called grey energy).

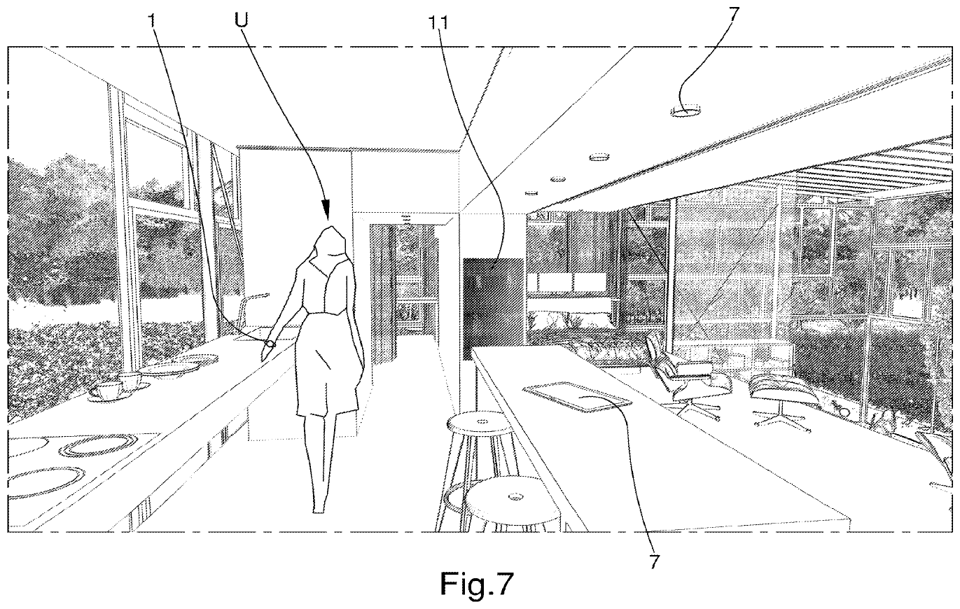

With reference to the example in FIG. 7, the monitoring system according to the invention first of all envisages the user U wearing at least a first detecting means 1 configured to detect first physiological parameters P1 of the user U. The first detecting means 1 may comprise e.g. a smart bracelet, a smart watch, a smartphone or similar smart devices capable of collecting at least one of the following parameters:
movement parameters by means of sensors such as: accelerometer, gyroscope, geomagnetic sensor, atmospheric pressure sensor, etc.,
biomedical parameters by means of sensors for measuring glucose, blood pressure, ECG, EMG, body temperature, heart rate, blood oxygen, blood pressure, sleep time, etc.,
environmental parameters such as temperature, humidity, gas, pH, ultraviolet, pressure, carbon dioxide, etc.,
technology use parameters such as e.g. internet connection usage times, entertainment usage times, etc.,
quality parameters related to the possessed/purchased items such as energy classes, origin, materials used, receivable through tags, QR code, etc.,

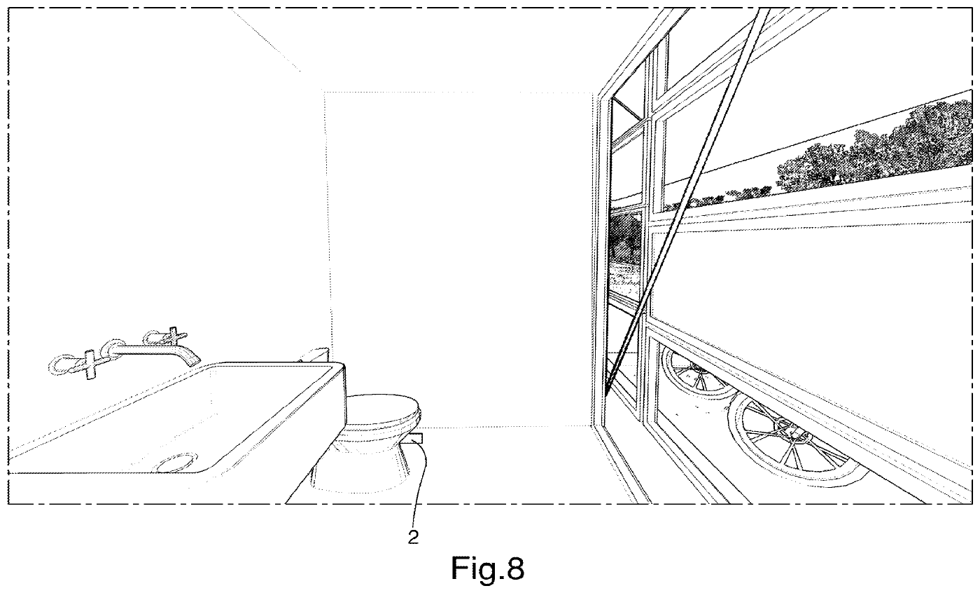

With reference to the example in FIG. 8, the system also envisages that inside the dwelling D of the user at least a second detecting means 2 is provided, configured to detect second physiological parameters P2 of the user corresponding to the liquid and solid evacuations produced by the user. In particular, the second detecting means 2 may comprise, e.g., an acquisition system as described in the publication dated Apr. 6, 2020 entitled “A mountable toilet system for personalized health monitoring via the analysis of excreta” by Seung-min Park et al, the contents of which are integrated herein as reference.

By way of example, the second physiological parameters P2 comprise parameters related to the user's vital values such as: protein, glucose, urine ph, liters of urine, etc.

Advantageously, the system has processing means M configured to receive the parameters P1, P2, process them and generate at least one food instruction I_{A} and/or dwelling instruction I_{D} optimized for the user.

In the remainder of the present description and in the subsequent claims, “food instruction” means one or more pieces of nutritional information suggested on the type of food the user should use to feed himself or herself according to the detected physiological parameters P1, P2 and other parameters which will be better specified in the remainder of the present description. Similarly, “dwelling instruction” means one or more pieces of information for the automatic and non-automatic coordination of the user points of the dwelling as well as units for the production of renewable energy such as heat, electricity and/or water.

Currently, the average energy demand (primary energy) per person in Europe is basically as follows:
1500 W for room air conditioning and hot water use,
1100 W for food (which includes transport),
600 W for electricity,
500 W for travel,
150 W for travel by public transport,
900 W for public infrastructure energy, giving a total of 5000 W and more of average annual energy requirement E.

As will be seen later in this disclosure, the parameters P1, P2 are used by the processing means M to calculate an average annual energy requirement E of the user U equal to or less than 2000 W (e.g. equal to 5.5 W per day or 2 kWh per hour or 48 kWh per day). To this end, one of the aims of the invention envisages that the dwelling D be completely self-sufficient in terms of food; it is therefore possible to have one or more plant organisms 3 or cultivable crops inside or outside the dwelling D. Such plant organisms 3 may comprise, e.g., fruit plants, able to provide not only sugary fruit but also complex carbohydrates, fats and proteins, berries with a triple attitude (fruit, biomass, nitrogen fixation), vegetables, other annual and biennial plants, carbohydrate and protein crops (potatoes, cereals, legumes) and other crops important for the self-supporting food and health of the user.

The design of the layout of the plant organisms 3 associated with the dwelling D is set up with the aim of minimizing processing and ensuring adequate health from the point of view of the phytosanitary profile of the crops as well as ensuring the best possible use of space without overloading the nutrient cycle in the soil with excessive drawdowns. In essence, feeding is compared and considered as an energy requirement of the building on a par with any other energy produced. The Applicant has ascertained that by means of a personal plant-based diet based on seasonal and home-grown products, it is possible to obtain a primary energy value of approx. 325 MJ/m² with expected greenhouse gas emissions of 12 kg/m².

Preferably, for each plant organism 3 or for each area containing one or more plant organisms, suitable detecting devices 4 can be provided, configured to acquire nutritional values P3 regarding, e.g., the growth and/or the state of maturity of the foods produced by them, their nutritional value, or even other readings such as soil pH, soil fertility (quantity of nitrogen, phosphorus, potassium, magnesium, calcium, sulfur), quantity of organic substance, etc. The value P3 of the foods will then be sent to the processing means M, according to known transmission techniques, in order to analyze them and coordinate them with the physiological parameters P1, P2 received from the same processing means M and consequently update the food instructions I_{A} and/or dwelling instructions I_{D} in order to optimize them.

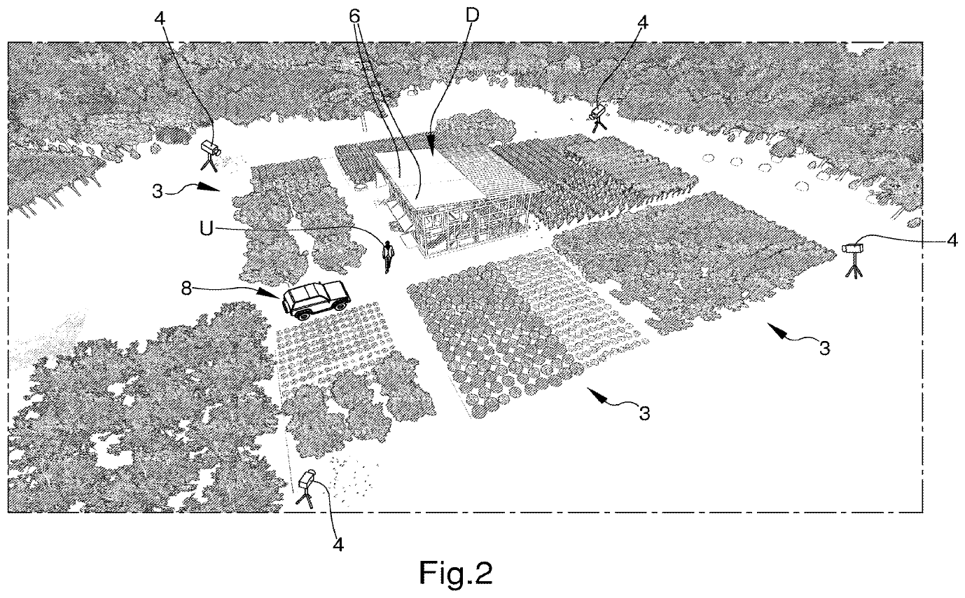

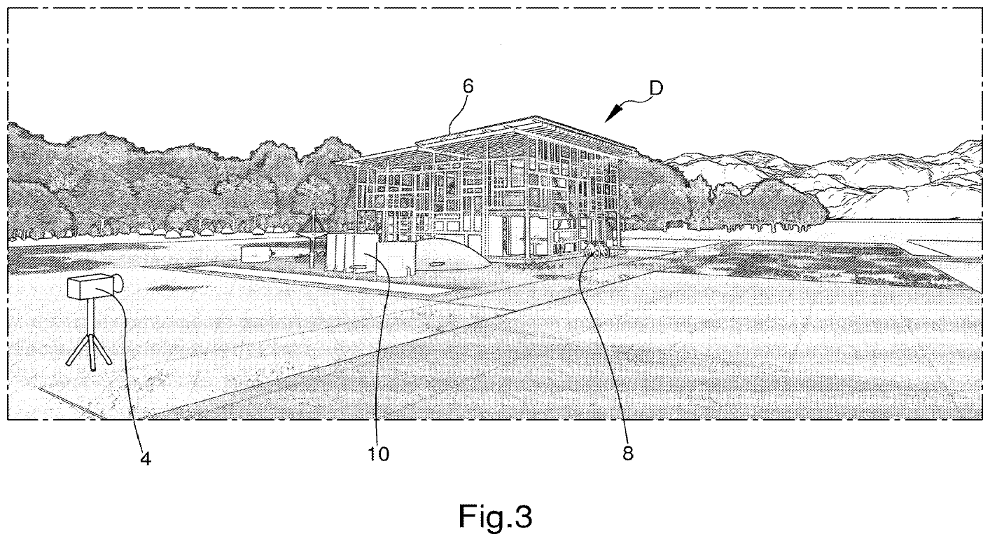

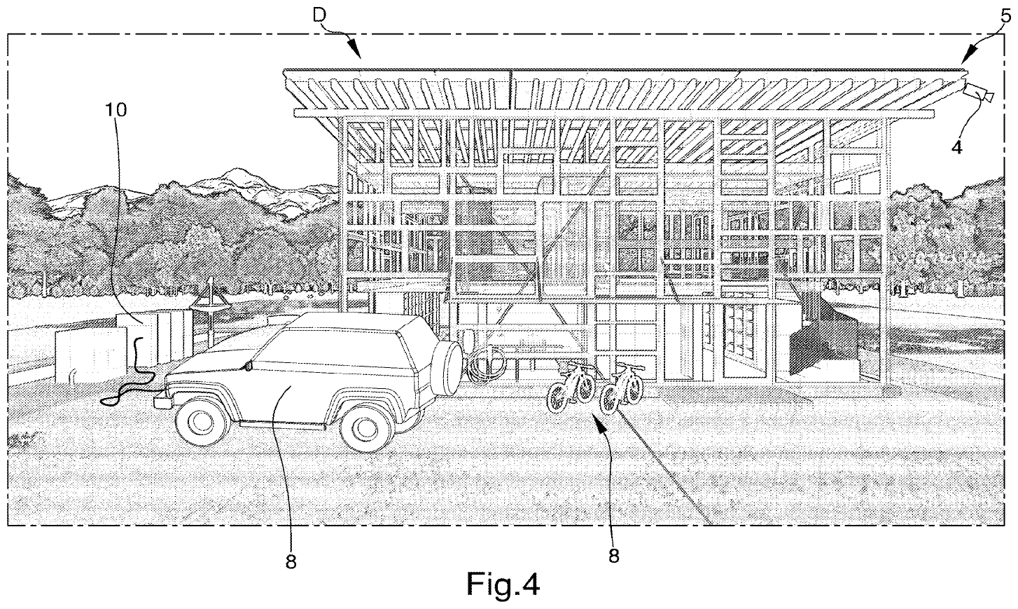

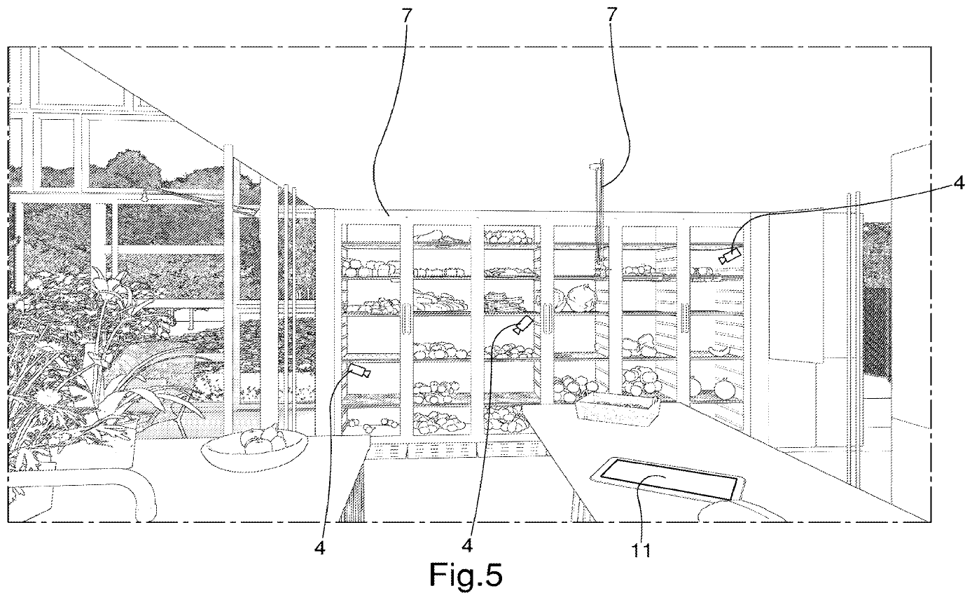

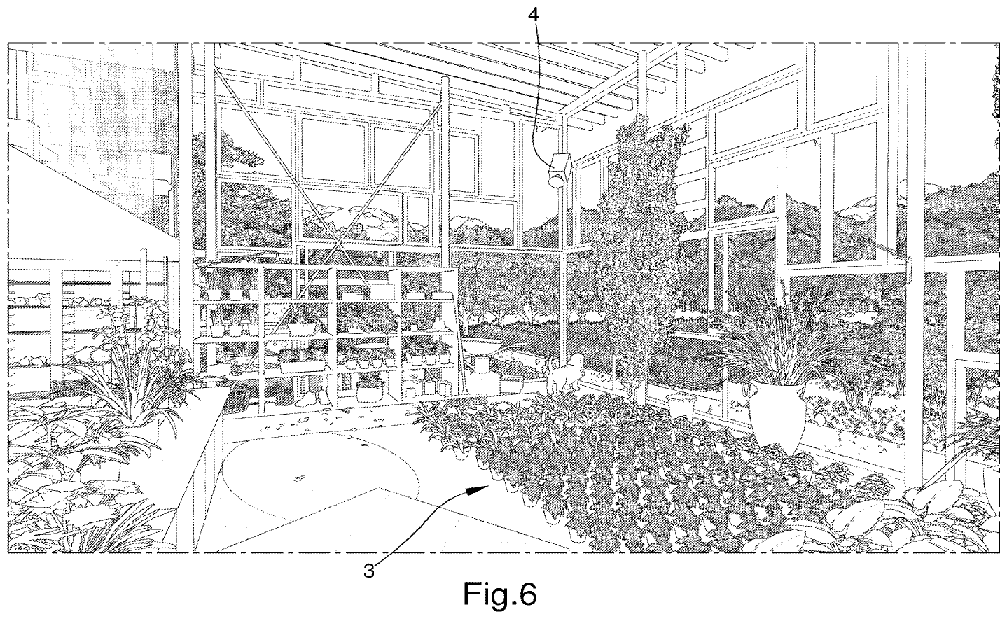

According to one version, the detecting devices 4 may comprise dedicated sensors such as, e.g., cameras, biosensors, etc. in order to acquire values P3. Conveniently, cameras may be used, either inside (FIG. 4) or outside (FIG. 2) the dwelling, to acquire visual information about individual fruit and vegetable products after harvesting. In this context, the processing means M can be configured to recognize the type of fruit and vegetable (e.g. using known image recognition techniques) and to estimate the nutritional value P3. The nutritional value P3 can e.g. be obtained and/or estimated during the storage of food in intelligent refrigerators (FIG. 5) connected to the processing means M. The estimated nutritional value P3 of the food will then be used by the processing means M to coordinate it with the physiological parameters P1, P2 and consequently update the food instructions I_{A} and/or dwelling instructions I_{D} to be given to the user U.

According to one embodiment, the system comprises at least one unit for the production of renewable energy 5. Preferably, such unit 5 may comprise devices for the production of energy by means of wind, geothermal, hydroelectric, marine, and/or solar systems. The unit for the production of renewable energy 5 may therefore comprise, e.g., solar/photovoltaic panels, water turbines, windmills, etc.

In the example shown in FIG. 2, unit for the production of renewable energy 5 comprises a plurality of photovoltaic panels 6 connected to an inverter for supplying power to the user points 7 (TV, lights, household appliances, etc.) of the dwelling according to per se known techniques. Conveniently, the inverter is in communication with the processing means M for the exchange of one or more electrical exchange values P4 corresponding to the production of the energy of the unit 5 and to the consequent absorption of the energy by the user points 7. The electrical exchange values P4 are sent to the processing means M and used by the latter to coordinate them with the physiological parameters P1, P2 and the nutritional values P3 in order to update the food instructions I_{A} and/or dwelling instructions I_{D} to be provided to the user U in an optimized manner

The electrical exchange values P4 may also comprise information about stored energy, energy produced by the panels, energy consumed, grey energy, etc. Additionally, the values P4 may comprise values of energy absorbed by the household appliances such as, e.g., absorption (in kWh), noise (dB), liters of water consumed, etc.

According to one embodiment, the user points 7 of the system may comprise at least one vehicle 8 of the smart type for the transport of the user U such as, e.g., a car, a bicycle, a motorbike, etc. The vehicle 8 comprises at least one control unit 9 for the calculation of the mobility values P5 such as, e.g., consumed electrical values, emissions, distances traveled, noise, etc., and for the communication of these values P5 to the processing means M using known transmission techniques, such as, e.g., transmission of the wireless type.

Preferably, the vehicle 8 is an electric type vehicle provided with its own internal rechargeable battery. The battery of the vehicle 8 is rechargeable via suitable charging station 10 associated with the dwelling D.

Conveniently, the mobility values P5 are sent to the processing means M and used by the latter to coordinate them with the physiological parameters P1, P2, the nutritional values P3 and the electrical exchange values P4 in order to update the food instructions I_{A} and/or dwelling instructions I_{D} to be provided to the user U in an optimized manner.

It should be observed that the food instructions I_{A} and/or dwelling instructions I_{D} to be given to the user are processed by the processing means M taking into account the average energy demand of approximately 2000 W of user U, as well as of the dwelling D, calculated over a period of one year. It may happen in fact that in some periods of the year, for example during the winter seasons, the average energy demand exceeds 2000 W. In this context, the processing means M will be configured to optimize consumption by balancing it over the year, e.g. by instructing the user U to consume less during summer periods.

Conveniently, the food instructions I_{A} may comprise indications as to the type of food the user should consume on a daily basis, e.g. through particular diets. In particular, the instructions I_{A} generated by the processing means M take into account both the food consumed by the user U by means of the productions from the plant organisms 3 of the dwelling D, and the food consumed by the user U not produced by such organisms 3. If, in fact, the user U dines out (e.g. in a restaurant), the user will send information about the food consumed to the processing means M by means, e.g., of a dedicated app installed on his or her smartphone.

According to a preferred embodiment, the system of the invention has a smart virtual assistant 11 in signal communication with the processing means M in order to interact with the user U. The assistant 11, by means of the processing means M, is able to receive information from all the devices installed therein such as, e.g., the detecting means and devices 1, 2, 4, the different units for the production of renewable energy 5, the user points 7, the vehicles 8, computers, notebooks, smartphones, etc. and to process the parameters P1-P5 thereof for the purpose of generating the food instructions I_{A} and/or the dwelling instructions I_{D}.

In one version, the processing means M are mounted on board the virtual assistant 11.

In a further version, the system may have a plurality of audio interfaces of the combined microphone/speaker type (not shown) in each room of the dwelling D, or also installed at predefined points outside, in signal communication with the personal assistant 11 in order to be able to acquire voice commands from the user U and emit the food instructions I_{A} and/or the dwelling instructions I_{D} in audible format.

In yet another version, the food instructions I_{A} and/or the dwelling instructions I_{D} can also be given through different interfaces, e.g. via the smartphone of the user U, in text format via dedicated apps, etc.

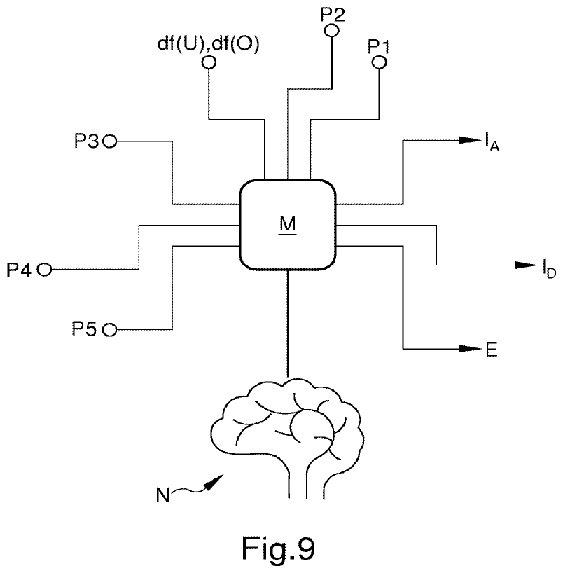

As shown in the example of FIG. 9, the system of the invention has at least one neural network N previously trained and in signal communication with the processing means M. As will be seen in the continuation of the present disclosure, the neural network N is configured to operate autonomously on the decision-making functions and to command the execution of predetermined machine learning algorithms associated with the processing means M.

In detail, the training of the neural network N is carried out through one or more preliminary phases of training used for the setting up of a training dataset through, first of all, an initial screening of the user U and of the dwelling D. Such screening allows acquiring a plurality of distinguishing characteristics df(U), df(D) that characterize the user U and the dwelling D. The training of the neural network N, aimed at learning the distinguishing characteristics df(U), df(D) as well as the information and properties associated with the user U and with the dwelling D, is carried out according to predefined machine learning algorithms. The training of the neural network N can be e.g. carried out through determinative algorithms (e.g. CNN, YOLO, SSD, etc.) able to learn not only the decision function but also the different representations of the distinguishing characteristics df(U), df(D) at different information levels.

In detail, in one of the preliminary training phases, there is a phase of providing a plurality of initial information about, e.g., the general health status of the user U and a plurality of properties/attributes concerning the devices and crops associated with the dwelling.

By way of example, the screening of the user U may comprise one or more of the following activities acquired at the beginning of the training phase:
medical examination,
collection of medical history data,
collection of anthropometric parameters (weight, height, body mass index, arm, waist, hip, thigh circumferences),
complete hematochemical examinations including blood glucose, glycated hemoglobin, insulin level, blood count with formula, serum iron, ferritin, total cholesterol, HDL, triglycerides, ALT, AST, γGT, FA, creatinine, TSH, vitamin B12, folic acid, homocysteine, vitamin 25-, OH D, parathormone, calcium, phosphorus, uric acid, potassium, sodium, chlorine, magnesium, PCR, zinc, copper, selenium, etc.,
complete urine and feces examination,
compilation of a 7-day food diary,
calculation of nutritional requirements,
drawing up a personalized food plan to achieve and/or maintain the user's ideal body weight,
working hours.

Subsequently, some of the above screening activities can be repeated at predefined monthly or annual intervals.

Similarly, the screening of the dwelling D allows defining a plurality of distinguishing characteristics df(D) taking into account at least one of the following characteristics:
features of the renewable energy production facilities (thermal, electrical and/or water energy),
features of the user points,
monitoring the evolution and stabilization of the crops for food production,
monitoring of climatic variability,
building characteristics such as, e.g., building construction energy values (SIA 2040), energy for possible building demolition, CECE-energy class building envelope, CECE-energy class building equipment,
control and measurement of data and integration with artificial intelligence such as, e.g., technology readiness level (TRS), business technology performance (BTP), artificial intelligence impact rate, digital integration rate, etc.,
building standards and design choices (SNBS, ECO, LEED-Minergie, Avoided Product).

Preferably, it must be specified that the characteristics df(D), df(U) related to the above listed characteristics are and may be selected and designed in a way to represent as much as possible qualitative technicalities always in step with the state of the art actually present on the market.

According to one version, the evolution and the state of the aspect related to food productions will be constantly monitored during the training phase and documented in order to produce adequate training of the neural network N on the productions to reach the prefixed objectives at the level of coverage of the food needs of the user U.

It has been estimated that a first phase of training can include a period of time equal to about three years during which the neural network N and, at the same time, the users U acquire knowledge in the field of horticulture, field-crops, processing, storage, etc. During this first phase, the soil, fertility in the broadest sense, and the various biological and microbiological balances of the natural system around the dwelling site will also be in the midst of a stabilization phase, so that the procurement of the distinguishing characteristics df(U), df(D) relating to this period will focus on monitoring the evolution and stabilization of the system rather than on actually achieving the maximum expected goals.

A second training phase which goes, e.g., from the third to the sixth year will make it possible to improve overall knowledge of the dwelling D as well as the agronomic knowledge of the soil so that the neural network N and consequently the user U achieve sufficiently consolidated learning. In this time frame, shrub and tree crops generally start to give satisfactory yields. In this second phase, the monitoring carried out by the processing means M guided by the neural network N will be of essential importance as it will allow generating food instructions I_{A} and/or dwelling instructions I_{D} for the user U which are increasingly more optimized in order to make possible a degree of energy and food self-sufficiency, thus consolidating the coordination between the values and the parameters P1-P5 acquired and constantly monitored.

A third phase of training, for instance from the sixth year on, will allow reaching a fine tuning level so that the food instructions I_{A} and/or dwelling instructions I_{D} generated by the processing means M, in association with the optimized decisional functions of the neural network N, allow calibrating the energy consumptions and the food productions to maintain an annual average energy requirement of about 2000 W within the target of 2000 W Society without affecting the wellbeing of the user U.

The above-described learning will allow the neural network N to assign, for each value and parameter P1-P5 and/or for each determined distinguishing characteristic df(U), df(D) acquired, a predefined accuracy threshold on the basis of the distribution of the analyses conducted during the preliminary training phases in order to minimize possible false positives/negatives while maintaining, at the same time, a high capacity to provide an immediate response during the subsequent phases of execution of the present invention, as will be seen later in the present disclosure.

From what has been said above, it is clear that the training is intended to set a training dataset containing all the information and the representative associations of the identified distinguishing characteristics df(U), df(D), as well as of the single parameters P1-P5, which are constantly saved in a database connected to the processing means M to train the neural network N.

It must be specified that already by the end of the procurement phase described above, it will be possible to interrogate the neural network N to command the execution of the processing means M and therefore generate one or more food instructions I_{A} and/or dwelling instructions I_{D} for the user U. Furthermore, during the entire training phase, the neural network N is both able to learn and to execute the instructions I_{A}, I_{D} in continuous mode. In point of fact, the neural network N improves the learning by adapting the execution and improving it as the training proceeds. In other words, the neural network N is substantially always in continuous training.

In summary, the system of the present invention provides for a plurality of training phases intended for the training of the neural network N which make it possible to automatically generate one or more food instructions I_{A} and/or dwelling instructions I_{D} optimized for the user U within the target of the 2000 W Society.

With reference to the examples shown in the attached illustrations, shown below are some of the possible phases of execution and generation of the food instructions I_{A} and/or dwelling instructions I_{D} according to the present invention.

In order to better understand the multitude of functions that the system of the invention can perform, the different operating conditions must be defined of the user U and/or of the dwelling D.

In relation to the user U, at least the following operating conditions can be defined, also combinable with each other:
eating condition by consumption of crops from the dwelling D,
eating condition by consumption of food not produced and grown in the dwelling D,
movement condition.

With reference instead to the dwelling D, at least the following operating conditions can be defined, also combinable with each other:
condition of renewable energy production
condition of energy withdrawal from user points;
condition of production of the products coming from the plant organisms 3,
condition of consumption of the products coming from plant organisms 3 by the user U.

By way of example, a typical day of an occupant/user U of the dwelling D could start by receiving a series of instructions suggested to him or her by the virtual assistant 11 comprising, e.g., dwelling instructions I_{A} (morning wakeup) and food instructions I_{D} (the food to be taken from the pantry for breakfast). The wakeup call will be set automatically by the neural network N on the basis of the habits of the user U acquired over time during the training, according to the statistical average rest requirement and age, etc. On the other hand, the instructions I_{A} concerning breakfast will also be suggested automatically by the virtual assistant on the basis of the training of the neural network N, depending on the food requirements of the user U, of the products available from the crops of the dwelling D, of the average annual energy requirement E, of the parameters P1-P5, etc.

Similarly, the mode of use of the vehicles 8, as well as of the user points 7 will be suggested by receiving one or more dwelling instructions I_{D} depending on the energy produced by the dwelling D, the average annual energy requirement E, the parameters P1-P5, etc.

If the user should have lunch or dinner outside the home, the virtual assistant 11, for example through his or her smartphone, will send a plurality of food instructions I_{A} on the type of food to be consumed always on the basis of the eating habits of the user U which the neural network N already knows. It follows that the latter will adapt its decisional functions by taking into account the annual average energy requirement E equal to or below 2000 W.

In virtue of the above, it has been ascertained that the calculation of the average energy requirement E can depend on different factors, for instance on:
a plurality of input-output information related to the generated learning dataset correlated to one or more conditions and/or phenomena under examination;
a plurality of input-output information related to the parameters P1-P5 acquired over time;
a plurality of input-output information related to the distinguishing characteristics df(U), df(D) acquired over time;
a plurality of input information representing the expected energy values (≤2000 W/capita);
a plurality of nutritional information representing the expected energy values (≤325 MJ/sqm).

In view of this, the neural network N is configured to generate one or a plurality of algorithms governing the conditions/phenomena under consideration on the basis of a predefined function constituted by the processing of all the information listed above.

As has been verified by the present description, it has been ascertained that the invention described herein achieves the intended objects and in particular the fact is underlined that by means of the system described herein it is possible to generate, with extreme accuracy, a plurality of food instructions I_{A} and dwelling instructions I_{D} for the user in a speedy automatic and optimized way within the scope of the 2000 W Society.

Furthermore, thanks to the innovative execution phase, it is possible to use the same instructions I_{A}, I_{D} to train the neural network N continuously over time. The effectiveness of the decisional functions of the neural network N thus gradually improves over time and speeds up the generation and effectiveness of the instructions I_{A}, I_{D}.

Finally, it should be noted that if the neural network N is not sure to provide an accurate result, it is always possible to question experts (doctors, nutritionists, etc.) so that the information of the neural network N can always be updated even with monitoring not acquired by the system.

Several tests have been carried out in order to simulate the dwelling conditions of a user and the supply of the optimized food instructions I_{A} and/or dwelling instructions I_{D} to the user to maintain an energy consumption of less than 2000 W per capita. The tests were carried out using the SimaPro software that allows collecting, analyzing and monitoring the environmental performance of products and services according to the ISO 14040-14044 standards. For the input of the values of the parameters P1-P5, df(U) and df(D), reference was made to some known data (e.g. nutritional values, energy consumption, physiological needs, etc.) while others were calculated using various databases, especially regarding environmental impacts such as:
raw materials;
input and output elements of all the manufacturing processes involved. “Input elements” are, e.g., the use of raw materials and energy resources. “Output elements” are, e.g.,
dissipated energy,
emissions into the air,
emissions into water,
waste,
distribution and transport,
production and use of fuels, electricity and heat,
use and maintenance of products,
disposal of waste and process products,
recovery of products after use,
manufacture of auxiliary materials
etc.

By way of example, the main databases used were Ecoinvent, USA Input Output Database, LCA Food DK, USLCI, ELCD, Industry data.

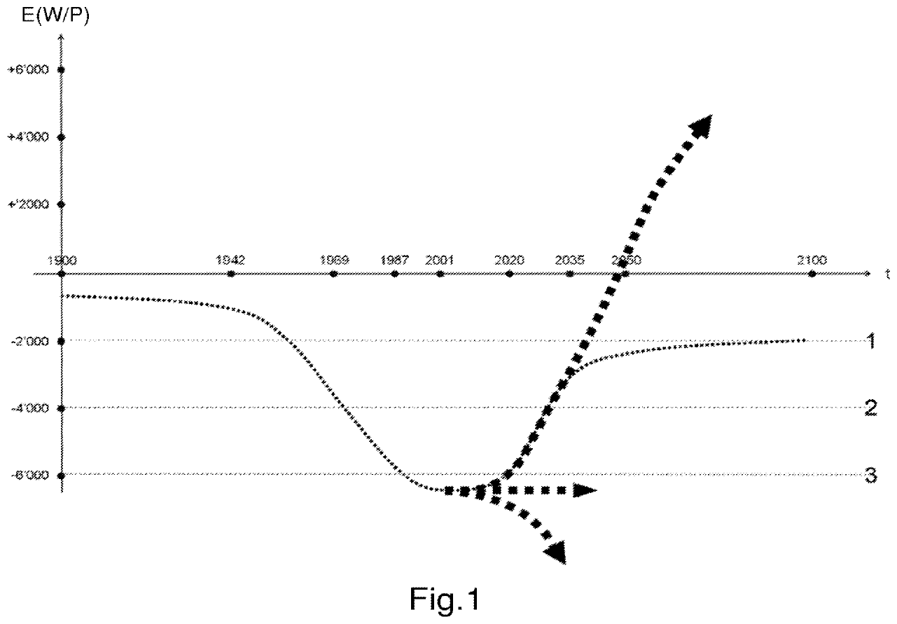

The tests made it possible to establish different user profiles and different instructions I_{A}, I_{D} which allow maintaining an average annual energy requirement E of about 2000 W per user. A planet/user sustainability ratio of one planet per 2000 W was also defined, as shown in the example in FIG. 1, which is able to show to the user in real time the sustainability ratio in terms of a score so that the same user can change his or her habits based on the trend of his or her consumption, as shown in the table below:

| Energy requirement E | No. of planets | Score |
|---|---|---|
| +2,000 | +1 | A |
| 0 | 0 | B |
| −2,000 | −1 | C |
| −4,000 | −2 | D |
| −6,000 | −3 | E |
| −8,000 | −4 | F |
| −10,000 | −5 | G |

A score of “A” indicates excellent behavior that would contribute to the sustainability of the planet, a score of “G” would indicate bad behavior whereby consumption would contribute to the exploitation, over time, of as many as five Earths. Thanks to the present invention, finally, it is possible to obtain a real climatic improvement by considering all the consumptions related to a user without neglecting any of them; the combination of the characteristics of the monitoring system as well as its execution are potentially endless and obviously a technician in the field, in order to satisfy contingent and specific needs, may make numerous modifications and variants, all of which are contained within the scope of protection of the invention, as defined by the following claims.

1. A system for monitoring the habits of one or more users, the system comprising:
at least one first detecting means wearable by a user to detect the first physiological parameters of the user,
at least one second detecting means configured to detect second physiological parameters corresponding to the liquid and solid evacuations of the user, and
processing means configured to receive said first and second parameters and to generate at least one user-optimized food instruction and/or dwelling instruction.

2. The system according to claim 1, further comprising: at least one neural network previously trained in signal communication with the processing means and configured to generate at least one optimized food instruction and/or dwelling instruction to be provided to the user.

3. The system according to claim 1, further comprising:
a dwelling unit,
least one plant organism, and
a detecting device configured to detect/estimate the nutritional values of the food produced by said at least one plant organism,
wherein said nutritional value is sent to the processing means to coordinate said nutritional value with said physiological parameters in order to update the food instructions and/or dwelling instructions to be provided to the user.

4. The system according to claim 1, further comprising:
at least one unit for the production of renewable energy, and
acquisition means for the acquisition of electrical exchange values corresponding to the production of energy of the unit and to the energy absorption by one or more user points of the dwelling,
wherein said electrical exchange values are sent to the processing means to coordinate them with said parameters in order to update the food instructions and/or dwelling instructions to be provided to the user.

5. The system according to claim 1, further comprising:
at least one vehicle provided with a control unit for the calculation of the consumed electrical mobility values for the communication of these values to the processing means,
wherein said electrical mobility values are sent to the processing means and used by the latter to coordinate them with said parameters in order to update the food instruction and/or dwelling instruction to be provided to the user in an optimized manner.

6. The system according to claim 1, wherein said processing means are configured to generate said food instruction and/or dwelling instruction depending on said parameters in order to estimate an average energy consumption of said user equal to or less than 2000 W per day.

7. A method for monitoring the habits of one or more users, said method comprising the following phases:
having a neural network in signal communication with processing means and configured to receive a plurality of parameters linked to said user and/or said dwelling according to claim 1,
setting a training dataset through an initial screening of the user and/or of the dwelling in order to acquire a plurality of distinguishing features of the user and of the dwelling,
generating at least one food instruction and/or dwelling instruction optimized to the user depending on said parameters and on said plurality of processed distinguishing features of the user and/or of the dwelling, and
estimating an average annual energy requirement for said user equal to or less than 2000 W.

8. The method according to claim 7, comprising the phase of:
using said instructions to train the neural network continuously over time in order to improve the decision-making functions of said network over time by speeding up the generation and effectiveness of said instructions to be provided to said user.

9. The method according to claim 7, comprising the phase of:
generating at least one machine learning algorithm that controls said food instruction and/or dwelling instruction, said parameters and of said plurality of distinguishing features in order to estimate an average annual energy requirement for said user equal to or less than 2000 W.

10. A system for monitoring the habits of one or more users, the system comprising:
at least one first detector configured to and/or programmed to detect the first physiological parameters of a user,
at least one second detector configured to and/or programmed to detect second physiological parameters corresponding to the liquid and solid evacuations of the user,
a processor or processing circuit configured to and/or programmed to receive said first and second parameters and configured to and/or programmed to generate at least one user-optimized food instruction and/or dwelling instruction.
